(19) United States
(12) Patent Application Publication
(10) Pub. No.: US 2026/0151647 A1
(43) Pub. Date: Jun. 4, 2026

(54) METHOD FOR CONTROLLING BACTERIAL BIOFILMS

(71) Applicant: Braincon GmbH & Co KG, Wien (AT)

(72) Inventor: Davul LJUHAR, Wien (AT)

(21) Appl. No.: 19/101,965

(22) PCT Filed: Aug. 5, 2023
(86) PCT No.: PCT/EP2023/071752

§ 371 Date: Jan. 5, 2026

(30) Foreign Application Priority Data
Aug. 8, 2022 (AT) A 60121/2022

(51) Int. Cl. A61N 5/06; A23B 2/00; A23B 2/53; A23B 2/783; A23B 2/788; A23B 2/792; A23B 4/015; A23B 4/22; A23B 4/24; A23B 4/30; A61K 35/76; A61L 2/102; A61L 2/104; A61L 2/22; A61L 103/05; A61L 103/06; B01L 1/02

(52) U.S. Cl. CPC A61N 5/0624; A23B 2/001; A23B 2/003; A23B 2/53; A23B 2/783; A23B 2/788; A23B 2/792; A23B 4/015; A23B 4/22; A23B 4/24; A23B 4/30; A61K 35/76; A61L 2/102; A61L 2/104; A61L 2/22; A61N 5/0616; B01L 1/025; A61L 2103/05; A61L 2103/06; A61L 2202/11; A61L 2202/121; A61L 2202/122; A61L 2202/14; A61L 2202/15; A61L 2202/16; A61N 2005/0626; A61N 2005/0661

(57) ABSTRACT

A method for combating bacterial biofilms by means of bacteriophages,
provision of a suspension containing one type of bacteriophages or a mixture of different types of bacteriophages or parts thereof,
provision of a treatment chamber (1), in which a human or animal carrier (2) medium is placed, and
non-destructive cold nebulization of the suspension of bacteriophages and introduction the generated mist into the treatment chamber (1) during a first treatment period so that the generated mist can act on the surface of the carrier medium (2).

The invention relates to a method for combating bacterial biofilms by means of bacteriophages.

In the past, a great deal of effort was put into combating bacterial biofilms and the microorganisms contained therein, in particular in connection with chronical wounds, food safety, and nosocomial infections, however, in many cases, the objectives pursued were only partly achieved or not achieved at all.

Also, treating biofilms with bacteriophages, e.g., in liquid form, has not met expectations until now.

It is thus an object of the invention to provide a method of the type mentioned above with greatly improved effects to allow, e.g., healing of chronical wounds.

According to the invention, this is achieved by
providing a suspension containing one type of bacteriophages or a mixture of different types of bacteriophages or parts thereof,
providing a treatment chamber, in which a human or animal carrier medium is placed, and
non-destructive cold nebulization of the suspension of bacteriophages and introducing the generated mist into the treatment chamber during a first treatment period so that the generated mist can act on the surface of the carrier medium.

Therefore, bacteriophages are used in a nebulized state for treating the surfaces of the carrier medium, in particular of lesions. The presence of a treatment chamber closed sealed off from the outside significantly increases the effect of the bacteriophages on the carrier medium because the bacteriophages contact the carrier medium in the form of a “dry” mist. By means of a nebulization technology (e.g., DCX), the bacteriophages could be successfully brought into the air without destroying their structure by mechanical stress, which was shown by transmission electron microscopy. It was possible to collect the bacteriophages-after nebulization-from a surface, e.g. a glass plate. The effect was proven in qualitative tests.

In addition, the bacteriophages were successfully deactivated by a treatment with H₂O₂ dry mist for 30 minutes.

It has been shown that this leads to a clearly detectable reduction of biofilms not only on wounds, etc., but also covers other application areas such as the preparation of food to ensure safe consumption, e.g., to prevent Salmonella infections.

In a further embodiment of the invention, cold nebulization can be conducted by means of a suitable device within the treatment chamber, by means of which problems related to positive pressure and excessive condensation of the cold mist can be solved.

According to a further exemplary embodiment of the invention, cold nebulization can alternatively also be conducted outside the treatment chamber, and the generated cold mist can be introduced into the treatment chamber from outside.

A further embodiment of the invention consists in introducing cold nebulized hydrogen peroxide, e.g. 1% to 40% H₂O₂, into the treatment chamber during a second treatment period after the first treatment period, so that the generated hydrogen peroxide mist can act on the surface of the carrier medium during the second treatment period.

This further increases the effect of the bacteriophage treatment combined with the H₂O₂ disinfection.

A further improvement of the effect of the hydrogen peroxide treatment can be achieved by at least temporarily irradiating the stream of cold nebulized hydrogen peroxide, which is introduced into the treatment chamber during the second treatment period, with UV light, which leads to an increase of hydroxyl radicals.

According to a further embodiment of the invention, the biofilm to be combatted can be present in the area of at least a part of the surface of the human or animal carrier medium.

According to a further embodiment of the invention, proteins of the bacteriophages can be used as particularly effective parts of the bacteriophages for cold nebulization and then be used for treating the carrier medium. Since the effect of the inactivation of the germs is mainly achieved via the proteins of the bacteriophages, it is possible to use only the cold nebulized form of the proteins for treating the carrier medium.

A further variation of the invention may consist in the fact that during the second treatment period, the surfaces pre-treated with cold nebulized bacteriophages are treated with a cold nebulized solution consisting of hydrogen peroxide with a content in the range of 1% to 40%, to which either no silver or silver or a silver compound with a content in the range of 5 ppm to 500 ppm and more has been admixed. The additional use of silver has, with certain germs, an enhancing effect in combatting germs.

Furthermore, it has proven advantageous to conduct an irradiation of the human or animal carrier medium with UVC before or after the cold nebulization step.

The size of the treatment chamber can be selected as desired, however, preferably it can have a volume in the range of 0.2 m³ to 100 m³.

In order to achieve the most efficient treatment of the carrier medium possible, the treatment chamber can be sealed off gas-tight to the outside after its introduction. This can be achieved by means of seals on housing parts or by means of sealing sleeves on limbs.

The carrier medium to be treated can, according to a further embodiment of the invention, be formed by a part of a limb of a living patient or an animal with a wound, since the inventive method has proven particularly useful for the treatment of biofilms forming in the area of wounds.

In a further embodiment of the invention, the carrier medium can be part of animal meat suitable for consumption. In this way, germs can be more efficiently combatted e.g. in the food industry.

The invention also relates to a treatment facility for conducting the inventive method, comprising:
a treatment chamber for placing the carrier medium,
a nebulizing device with a port for introducing a liquid to be nebulized and with an outlet for expelling the mist into the interior of the treatment chamber.

In order to not release the expelled mist into the environment without further use after it has flowed through the treatment chamber, the nebulizing device can, in a further development of the invention, have at least one inlet for reintroducing the mist from the treatment chamber.

It has been shown to be advantageous if, according to a further embodiment of the invention, the nebulizing device is formed as a portable unit that is introducible into the interior of the treatment chamber, the generated mist within the treatment chamber exiting via the at least one outlet and, after flowing through the treatment chamber, reentering the nebulizing device via the at least one inlet.

The nebulizing device may also be permanently arranged within the treatment chamber, however, arranging a portable nebulizing device in the treatment chamber makes its cleaning substantially easier and the nebulizing device can also be quickly replaced by another nebulizing device nebulizing a different liquid, e.g., a suspension of bacteriophages or hydrogen peroxide.

A particularly advantageous design of the nebulizing device may consist in the nebulizing device comprising an inner housing box with at least one nebulizing unit, the at least one inlet, the at least one outlet, and a control, as well as an outer trough into which the inner housing box is inserted.

This provides for very good protection and easy cleaning of the components. Liquids can be fed from the interchangeable outer troughs, e.g., an outer trough with bacteriophages and an outer trough with H₂O₂.

In a further embodiment of the invention, the port for a liquid to be nebulized may be connectable to a first container containing a suspension containing one type of bacteriophages or a mixture of different types of bacteriophages or their parts. Depending on the type of treatment, the composition of the suspension to be nebulized can thus be changed very easily.

In particular, the port can be connectable to a second container for a liquid to be nebulized, which contains hydrogen peroxide (H₂O₂) or a solution thereof, and wherein the port can optionally also be connectable to an O₂ source. Neither the number nor the content of the containers for the liquid to be nebulized are in any way limited within the scope of the invention.

The type of cold nebulization may also vary. Is has proven to be very advantageous if the nebulizing device produces an aerosol with a droplet distribution with a maximum droplet diameter substantially within the range of 0.1 μm to 10 μm. This leads to less humid precipitation, e.g. on the walls, which should be avoided as much as possible in order to not reduce the efficiency of the inventive method.

Furthermore, an intermediate pipe section or an attachment may be within the inventive facility, which has UV lamps or LED elements on the interior walls for irradiating the mist flowing through it, which is connected to the outlet of the nebulizing device.

An irradiation device may also be provided in the treatment chamber, so that according to a further exemplary embodiment, a UVC irradiation device may be arranged for irradiating the carrier medium.

In a further embodiment of the invention, the outlet of the nebulizing device may be provided with the attachment lined with an UV-A, -B, or -C element, which irradiates the exiting mist with a wavelength between 70 nm and 400 nm, which leads to the generation of additional free radicals, i.e., highly reactive hydroxyl radicals.

According to a further exemplary embodiment, the concentration of H₂O₂ and/or OH can be increased after 5 min and rise continuously.

According to a further exemplary embodiment, an attachment may be provided that also irradiates exiting gas, but can optionally simultaneously irradiate the surroundings. A further embodiment of the invention may consist in a device inactivating the germs optionally with UVB or UVC (for human subjects) with a wavelength below 220 nm by means of a device especially developed for treating surface germ contaminations, wherein the device can be manually activated or machine-controlled.

A further improvement of the inventive facility in the field of food production can be achieved by integrating the UVB or UVC elements into respective locks.

Below, the invention will be explained in more detail by means of exemplary embodiments. Therein,

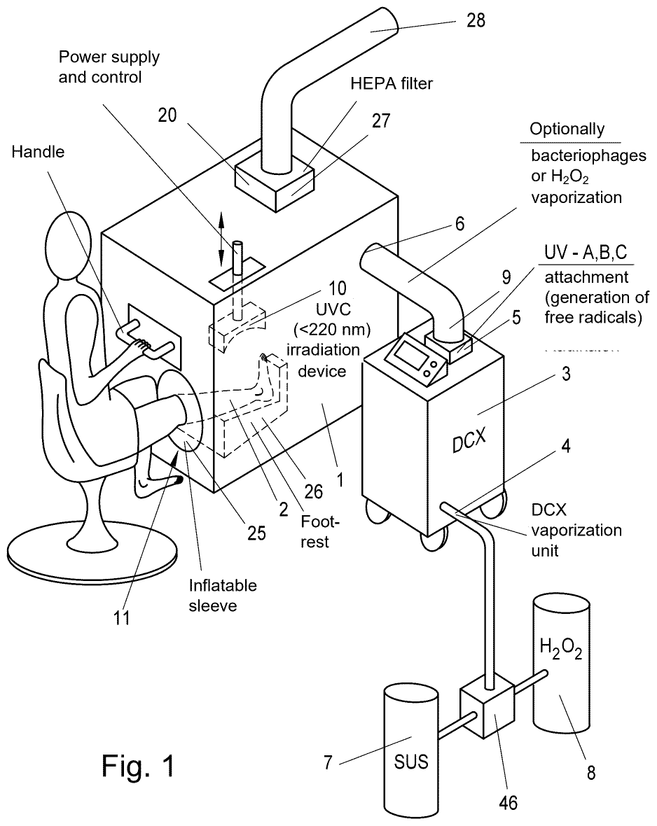

FIG. 1 shows a sketch of the principle of a first embodiment of an inventive facility for using the inventive method;

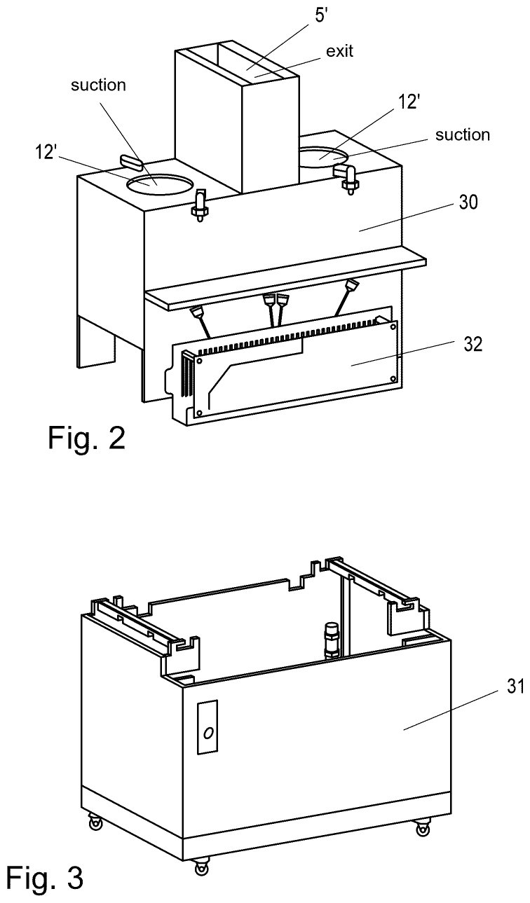

FIGS. 2 and 3 show an explosive view of an embodiment of a nebulizing device of the inventive facility;

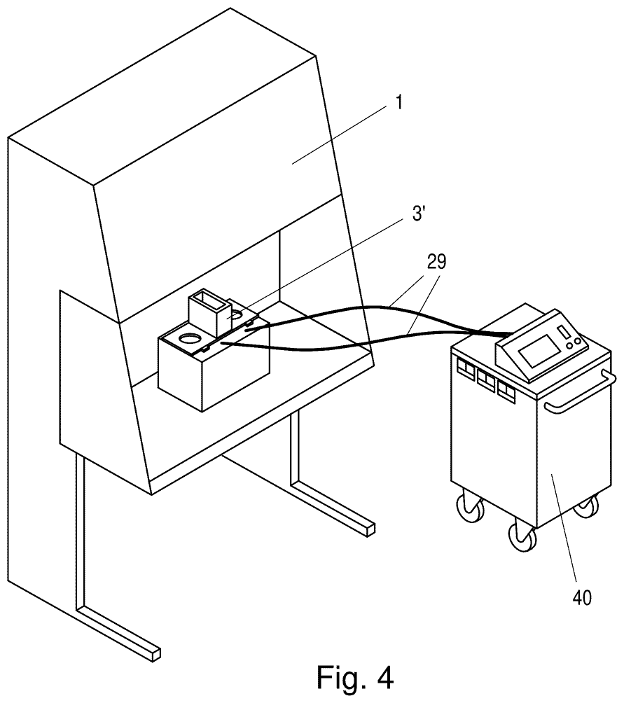

FIG. 4 shows an angular view of a further embodiment of an inventive facility for using the inventive method;

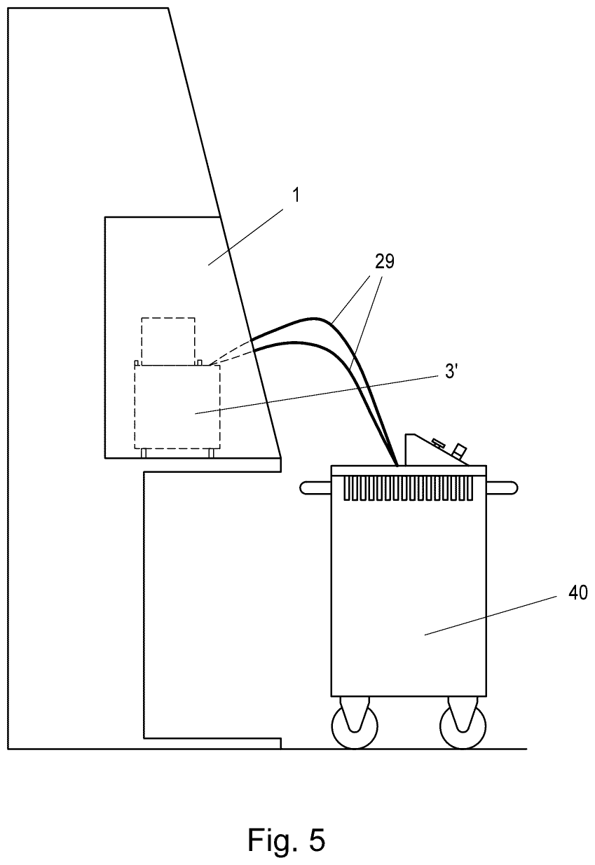

FIG. 5 shows a side view of the embodiment of FIG. 4;

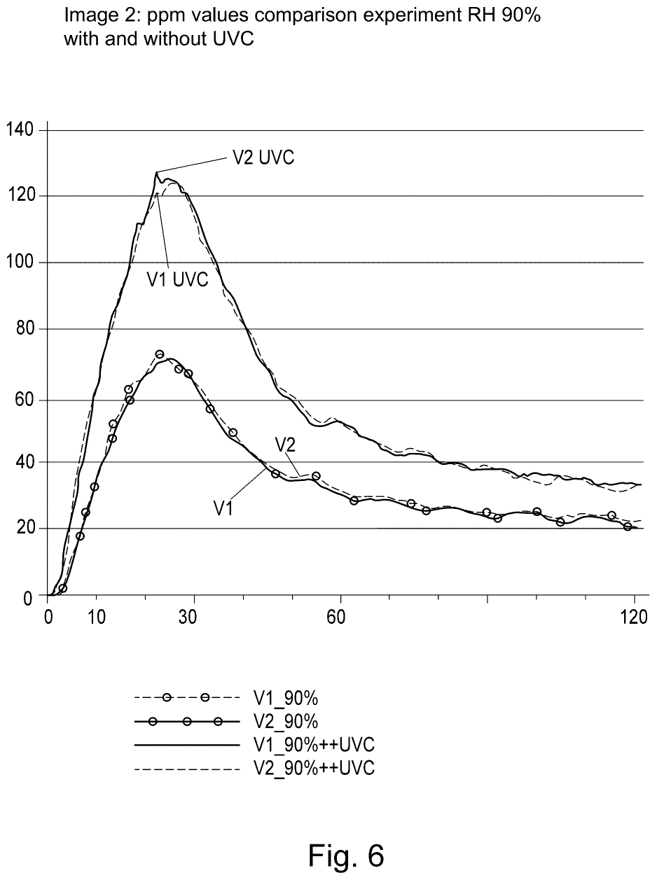

FIG. 6 shows a plot of the achievable concentration of hydrogen peroxide in the treatment chamber over time;

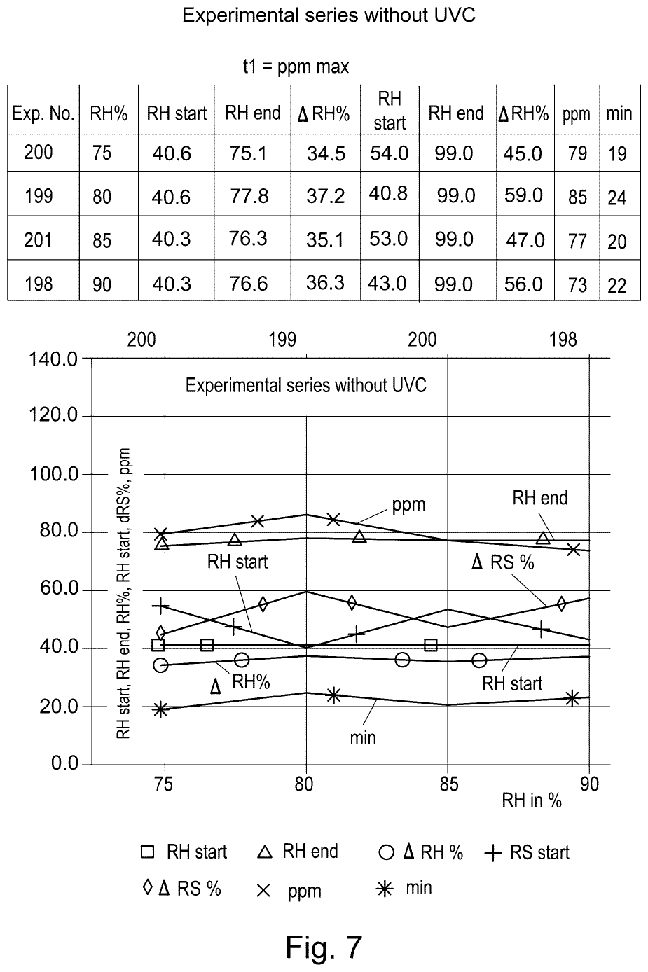

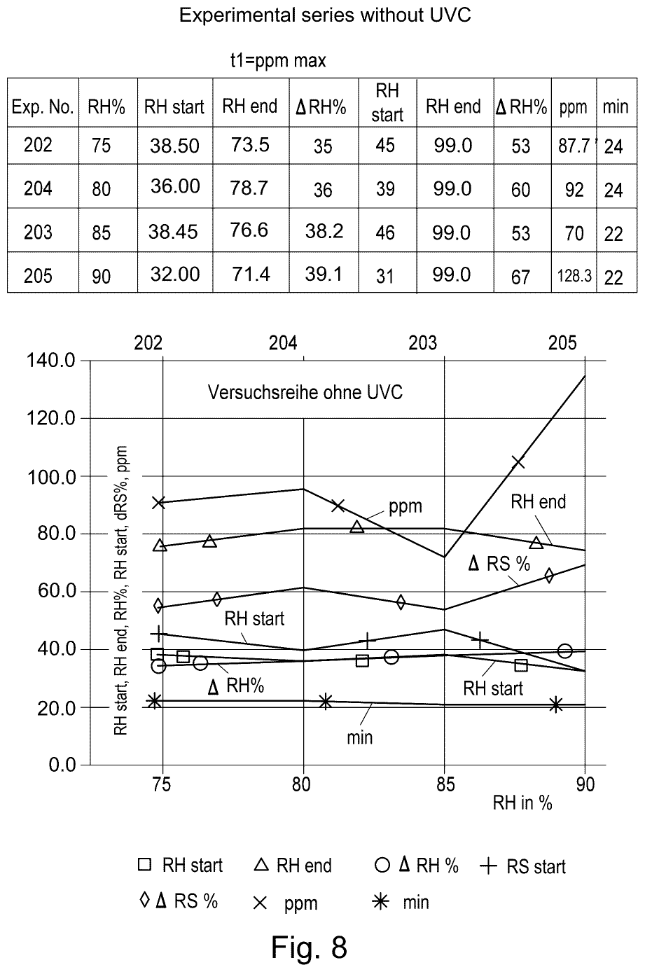

FIGS. 7 and 8 each show a graph of the peroxide concentration in experimental series with and without UVC;

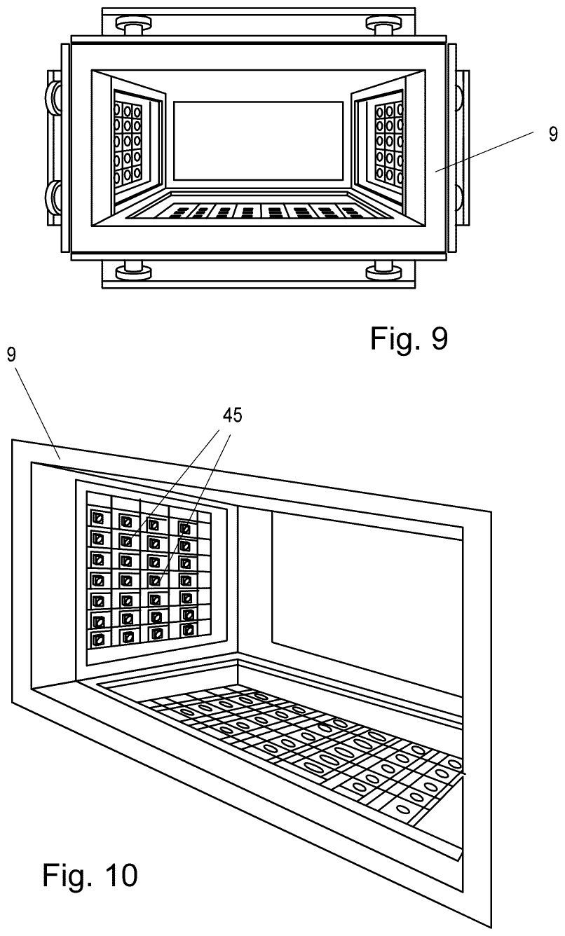

FIGS. 9 and 10 show angular views of an embodiment of a UVC attachment;

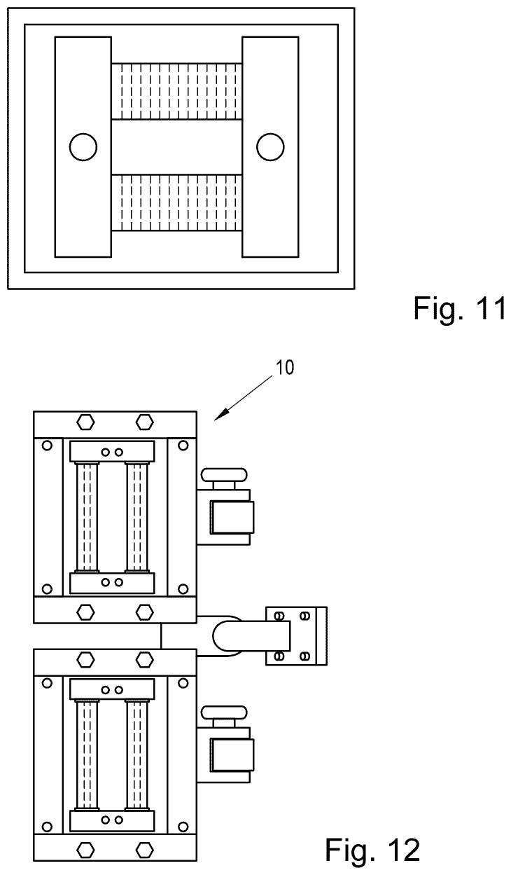

FIG. 11 shows a plan view of a further embodiment of the UVC attachment;

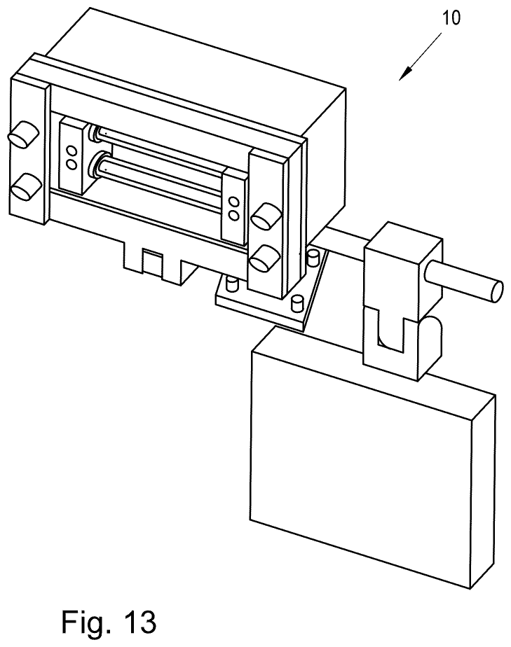

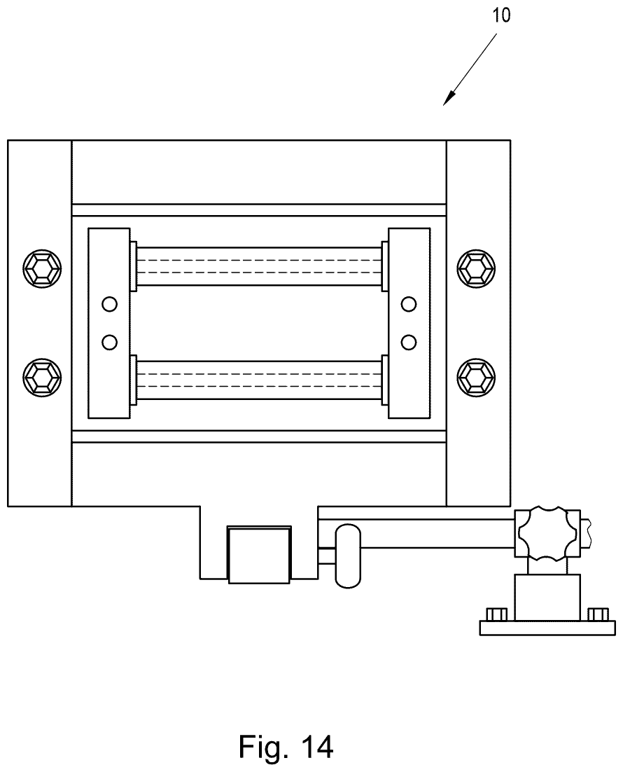

FIGS. 12, 13 and 14 show plan and angular views of an embodiment of an irradiation device;

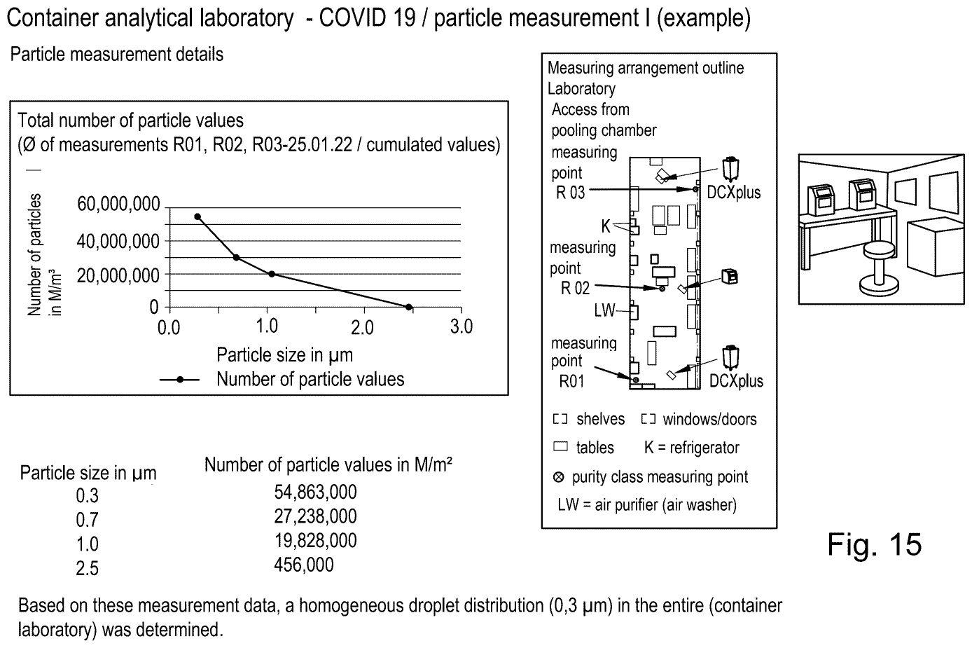

FIG. 15 shows a plot of measured particle size values in the treatment chamber; and

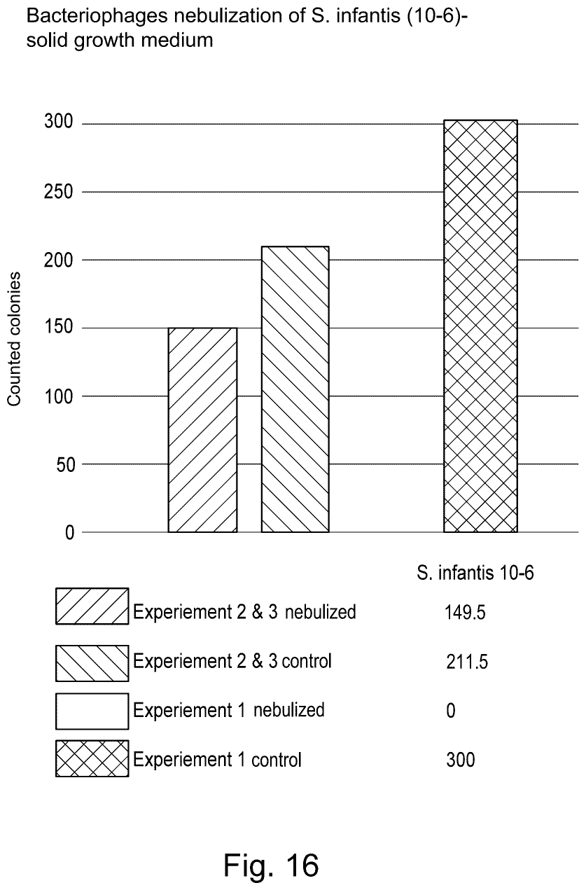

FIG. 16 shows a measurement graph of the nebulization of bacteriophages within the treatment chamber.

FIG. 1 shows a first embodiment of a treatment chamber 1 serving for treating chronical wounds and lesions respectively, e.g., on limbs, e.g. on legs or feet.

The situation is that the biofilm to be combatted is present in the area of at least a part of the surface of the human carrier medium. The inventive method is substantially composed of the following steps:
providing a suspension containing one type of bacteriophages or a mixture of different types of bacteriophages or parts thereof,
providing a treatment chamber 1, in which a human or animal carrier medium 2 is placed, and
non-destructive cold nebulization of the suspension of bacteriophages and introducing the generated mist into the treatment chamber 1 during a first treatment period so that the generated mist can act on the surface of the carrier medium 2.

The carrier media treatable with the inventive method may be from human or animal origin. The first treatment period can be freely selected depending on the type of bacteriophages, however, is preferably in the range of several minutes.

FIG. 1 shows an embodiment of the invention in which the carrier medium 2 is formed by part of a limb of a living patient with a wound, which may, however, also be an animal, wherein the treatment of animals is also possible in a dead state, e.g., for combatting Salmonella. In particular, the carrier medium may be a piece of animal meat suitable for consumption.

There is no particular limitation to a certain type of bacteriophages or mixtures of bacteriophages to be used. The bacteriophages used may be from one or several groups or their parts, e.g., proteins.

The suspension of bacteriophages used for cold nebulization can, without limitation, be formed based on a lytic Salmonella infantis bacteriophage culture, the concentration being 10⁻⁸.

The results of the S. infantis bacteriophages are similar to Campylobacter or E. coli, S. aureus bacteriophages, which have also been used in experiments. They are true for all bacteriophages because the same mechanisms always come into play. The method also has such an effect with virophages. FIG. 16 shows an example of the measured nebulization of bacteriophages in an experimental series with S. infantis, the Y axis showing the colonies counted.

The treatment facility for conducting the inventive method comprises
the treatment chamber 1 for introducing the carrier medium 1,
a nebulizing device 3 with a port 4 for a liquid to be nebulized and with an outlet 5, which is connected to the inlet 6 of the treatment chamber 1.

Preferably, the treatment chamber 1 defines a volume of a cavity in the range of 0.2 m³ to 100 m³ , without being limited thereto.

After introducing the human or animal carrier medium 2, the treatment chamber 1 is, in the shown example according to FIG. 1, preferably sealed off gas-tight to the outside. For this purpose, the treatment chamber 1 has a means 11 for gas-tight sealing after introduction of the carrier medium 2, here, a rubber sleeve 25 through which the limb is passed and which is supported on a foot support 26 in the interior of the treatment chamber 1.

After the first treatment period, a second treatment period, which preferably lasts several minutes, comprises the introduction of cold-nebulized hydrogen peroxide into the treatment chamber 1 so that the mist generated during the second treatment period can act on the surface of the carrier medium 2.

The flow of cold-nebulized hydrogen peroxide, which is introduced into the treatment chamber 1 during the second treatment period, is irradiated with UV light for at least part of the time. This irradiation is not obligatory and can be omitted, however, it increases the effect.

Furthermore, an intermediate pipe section (FIGS. 9 and 10) or an attachment 9 is provided for this purpose, which has an UV lamp (FIG. 11) or UV LED elements 45 (FIG. 10) on its interior wall for irradiating the mist flowing therethrough, which is interposed between the outlet 5 of the nebulizing device 3 and the inlet 6 of the treatment chamber 1.

The outlet 5 of the nebulizing unit 3 provided with the attachment 9 is lined with the UV LED elements 45, e.g., UV-A, -B or -C elements, which irradiate the exiting mist with a wavelength between 70 nm and 400 nm, which leads to the generation of additional free radicals, i.e., highly reactive hydroxyl radicals.

Furthermore, an UVB/UVC irradiation device 10 (FIGS. 12, 13 and 14) for irradiating the carrier medium 2 is arranged in the interior of the treatment chamber 1. Thus, an irradiation of the human or animal carrier medium with UVB/UVC can be conducted before or after the cold nebulizing step during an irradiation period. By means of the irradiation device 10 particularly developed for treating surface germ contaminations, germs in the interior of the treatment chamber 1 can be inactivated optionally with UVB or UVC (for human subjects) with a wavelength below 220 nm, wherein the irradiation device 10 can be manually activated or machine-controlled. In the field of food production, the UVB or UVC elements can be integrated in respective locks.

In addition, it is possible to treat the surfaces pre-treated with a bacteriophage mist, during the second treatment period, with another nebulized liquid consisting of hydrogen peroxide with a content in the range of 1% to 40%, to which either no silver or silver or a silver compound with a content in the range of 5 ppm to 500 ppm (and more) has been admixed.

The port 4 for a liquid to be nebulized is connectable to a first container 7 containing a suspension that contains one type of bacteriophages or a mixture of different types of bacteriophages or their parts.

The port 4 for a liquid to be nebulized is connectable to a second container 8 containing hydrogen peroxide (H₂O₂) or a solution thereof via a switching valve 46, so that the port 4 can additionally be connected with an H₂O₂ source for the conduction of the optional treatment with hydrogen peroxide.

A specific feature of the inventive method is the nebulization of the suspension of bacteriophages, wherein the nebulization device 3 generates an aerosol with a droplet distribution with a maximum droplet diameter substantially within the range of 0.1 μm to 10 μm. Here, FIG. 15 shows an example of a measured droplet size distribution in the treatment chamber 1.

An outlet 20 of the treatment chamber 1 is provided with a filter 27 for pressure compensation because the inflowing cold mist can easily lead to positive pressure. The outgassed vapors can also be reintroduced into the nebulization device 3 or they escape via an exhaust pipe 28 to the surrounding provided.

One embodiment of the method consists in the fact that the concentration of H₂O₂ and/or OH and/or O₂ is increased after 5 minutes and rises continuously.

Furthermore, an attachment is provided that also irradiates exiting gas, but at the same time irradiates the surrounding.

FIGS. 2, 3, 4 and 5 show a nebulizing device 3′of a further exemplary embodiment of the invention. In this exemplary embodiment, there is no positive pressure in the treatment chamber because the nebulizing device 3′is formed as a portable unit that is introducible into the interior of the treatment chamber 1, the generated mist inside the treatment chamber 1 exiting via the one outlet 5′and, after flowing through the treatment chamber 1, reentering the nebulization device 3′via two inlets 12′. This leads to a circulation through the nebulization device 3′, which avoids positive pressure. During operation, a control panel 40 is connected to the nebulization device 3′arranged in the treatment chamber 1 via connecting lines 25.

FIGS. 2 and 3 show the internal structure of the nebulizing device 3′, which comprises an inner housing box 30 with at least one nebulizing unit (not shown) in the interior, the inlets 12′, the outlet 5′, and a control 32, as well as an outer trough 31 into which the inner housing box 30 is inserted.

The outer trough 31 serves for receiving a liquid to be nebulized, e.g., a suspension of bacteriophages or a hydrogen peroxide solution, and can be easily replaced and cleaned.

FIG. 6 shows a plot of the H₂O₂ concentration in the treatment chamber 1 during the second treatment period. The X axis represents the time axis, i.e., 120 min were measured. The Y axis represents the H₂O₂ concentration in ppm. The measurements were conducted with the Vaisala System. Two measurement systems V1 and V2 were always used parallel.

The upper curves V1, V2 represent the H₂O₂ concentration in ppm, at a relative humidity of 90% and with the use of the UVC(UVB) attachment 9. The lower curves V 1, V 2 represent the corresponding values without UVC(UVB) attachment 9.

FIG. 8 shows the increase in the H₂O₂ concentration in ppm in relation to the RH (relative humidity) in % and the use of the UVC(UVB) attachment 9. Higher RH values and longer vaporization times lead to higher ppm concentrations. FIG. 7 shows the values without UVC(UVB) attachment 9.

1.-28. (canceled).

29. A method for combating bacterial biofilms by means of bacteriophages, comprising:
providing a suspension containing one type of bacteriophages or a mixture of different types of bacteriophages or parts thereof,
providing a treatment chamber (1), in which a human or animal carrier (2) medium is placed, and
non-destructive cold nebulization of the suspension of bacteriophages and introducing the generated mist into the treatment chamber (1) during a first treatment period so that the generated mist can act on the surface of the carrier medium (2).

30. The method according to claim 29, wherein the cold nebulization is conducted within the treatment chamber (1) or
the cold nebulization is conducted outside the treatment chamber (1) and the generated mist is fed into the treatment chamber (1) from outside and
cold nebulized hydrogen peroxide, e.g. 1% to 40% H₂O₂, is introduced into the treatment chamber (1) during a second treatment period after the first treatment period, so that the generated hydrogen peroxide mist can act on the surface of the carrier medium (2) during the second treatment period.

31. The method according to claim 29, wherein the flow of cold-nebulized hydrogen peroxide, which is introduced into the treatment chamber (1) during the second treatment period, is irradiated with UV light for at least part of the time.

32. The method according to claim 29, wherein the biofilm to be combatted is present in the area of at least a part of the surface of the human or animal carrier medium.

33. The method according to claim 29, wherein the parts of the bacteriophages are proteins.

34. The method according to claim 29, wherein bacteriophages from one or more groups or their proteins are used.

35. The method according to claim 30, wherein during the second treatment period, the surfaces pre-treated with cold nebulized bacteriophages are treated with a cold nebulized solution consisting of hydrogen peroxide with a content in the range of 1% to 40%, to which either no silver or silver or a silver compound with a content in the range of 5 ppm to 500 ppm and more has been admixed, and
the conduction of an irradiation of the human or animal carrier medium with UVC before or after the cold nebulization step during an irradiation time.

36. The method according to claim 29, wherein the carrier medium (2) is formed by a part of a limb of a living patient or an animal with a wound.

37. The method according to claim 29, wherein the carrier medium is a piece of animal meat suitable for consumption.

38. A treatment facility for conducting the method according to claim 29, comprising:
a treatment chamber (1) for placing the carrier medium (2),
a nebulizing device (3, 3′) with a port (4) for providing a liquid to be nebulized and with an outlet (5, 5′) for expelling the mist into the interior of the treatment chamber (1).

39. The facility according to claim 38, wherein the nebulizing device (3′) is formed as a portable unit that is introducible into the interior of the treatment chamber (1), the generated mist within the treatment chamber (1) exiting via the at least one outlet (5′) and, after flowing through the treatment chamber (1), reentering the nebulizing device (3) via the at least on inlet (12′).

40. The facility according to claim 39, wherein the nebulizing device (3′) comprises an inner housing box (30) with at least one nebulizing unit, the at least one inlet (12′), the at least one outlet (5′) and a control (32), as well as an outer trough (31) inserted into the inner housing box (30).
and the port (4) for a liquid to be nebulized is connectable to a first container (7) containing a suspension containing one type of bacteriophages or a mixture of different types of bacteriophages or their parts.

41. The facility according to claim 38, wherein the port (4) for a liquid to be nebulized is connectable to a second container (8), which contains hydrogen peroxide (H₂O₂) or a solution thereof, and that the port (4) is optionally also connectable to an O₂ source. and
the treatment chamber has means (11) for gas-tight sealing after introduction of the carrier medium (2).

42. The facility according to claim 38, wherein the nebulizing device (3, 3′) produces an aerosol with a droplet distribution with a maximum droplet diameter substantially within the range of 0.1 μm to 10 μm.

43. The facility according to claim 38, wherein an intermediate pipe section or an attachment (9) is provided, which has UV lamps or LED elements (45) on the interior walls for irradiating the mist flowing through it, which is connected to the outlet (5) of the nebulizing device (3).

44. The facility according to claim 38, wherein a UVC irradiation device (10) is arranged in the treatment chamber (1) for irradiating the carrier medium (2) and -the outlet (5) of the nebulizing device (3) is provided with the attachment (9) lined with an UV-A, -B, or -C element, which irradiates the exiting mist with a wavelength between 70 nm and 400 nm, which leads to the generation of additional free radicals, e.g., highly reactive hydroxyl radicals.

45. The facility according to claim 38, wherein the concentration of H₂O₂ and/or OH and/or O₂ is increased after 5 min and rises continuously.

46. The facility according to claim 43, wherein an attachment is provided that also irradiates exiting gas, but can optionally simultaneously irradiate the surroundings.

47. The facility according to claim 38, wherein the germs are inactivated optionally with UVB or UVC (for human subjects) with a wavelength below 220 nm by means of a device especially developed for treating surface germ contaminations, wherein the device can be manually activated or machine-controlled.

48. The facility according to claim 38, wherein in the field of food production, the UVB or UBC elements are integrated into respective locks.
